(19) United States
(12) Patent Application Publication
(10) Pub. No.: US 2022/0228192 A1
(43) Pub. Date: Jul. 21, 2022

(54) Method To Evaluate Metabolic Activity Of Liver Enzymes

(71) Applicant: Owlstone Medical Limited, Cambridge (GB)

(72) Inventors: Edoardo Gaude, Cambridge (GB); Max Allsworth, Cambridge (GB); Marc van der Schee, Cambridge (GB)

(21) Appl. No.: 17/055,914

(22) PCT Filed: May 17, 2019
(86) PCT No.: PCT/GB2019/051381

§ 371 Date: Nov. 16, 2020

(30) Foreign Application Priority Data
May 17, 2018 (GB) 1808062.2

(51) Int. Cl. C12Q 1/26; G01N 33/50

(52) U.S. Cl. CPC C12Q 1/26; G01N 33/5038; G01N 33/5014; G01N 2407/00; G01N 2800/085; G01N 2800/52; C12Y 114/14

(57) ABSTRACT

The invention provides a breath test for assessing a metabolic phenotypeand/or assessing a disease state.

### RELATED APPLICATION(S)

This application is the U.S. National Stage of International Application No. PCT/GB2019/051381, filed May 17, 2019, which designates the U.S., published in English, and claims priority under 35 U.S.C. § 119 or 365(c) to Great Britain Application No. 1808062.2, filed May 17, 2018. The entire teachings of the above applications are incorporated herein by reference.

### INTRODUCTION

Different individuals react differently when exposed to the same exogenous substances at the same dosage. In day to day life, this is most apparent and relevant in case of bodily exposure to (potentially) toxic and or beneficial substances, for example so-called xenobiotics, such as therapeutic drugs or pollutants. These differences are a direct consequence of differences in the way the body interacts with these compounds through absorption, distribution, metabolism and excretion, so-called pharmacokinetics. The efficiency with which the body metabolises a drug into its inactive form or a pro-drug into its active form is a key determinant of both the efficacy and toxicity of such compounds. The cytochrome CYP450 (CYP450) enzyme family is responsible for metabolism of most drugs and lipophilic xenobiotics, and are therefore of great importance for clinical pharmacology. Although several different families of CYP450 enzymes are present in the human body, the enzymes belonging to 1-, 2-, and 3-families are involved in the metabolism of the great majority of administered therapeutic drugs (Zanger and Schwab, 2013).

These enzymes transform pro-drugs into corresponding bioactive compounds, as well as active drugs into inactive metabolites that are subsequently excreted from our body. Differences in enzymatic activity of these enzymes lead to different biotransformation of xenobiotics, ultimately resulting in toxicity of a compound or inefficacy of a drug. Many factors contribute to the diversity in CYP450s activity and consequently their metabolic efficiency. Assessing the metabolic phenotype of CYP450 enzymes is critical to detect and predict the biological variability of metabolic efficiency of xenobiotics, ultimately to prevent potential adverse reactions and maximise the efficiency.

Currently, genetic tests are available to assess genetic polymorphisms that could result in fast or slow metabolism of xenobiotics (Ned Mmsc Phd, 2010; Thakur et al., 2007). These tests have been developed for the assessment of CYP450-specific genotypes, including CYP1A, 2A6, 2B6, 2C19, 2C8/9, 2D6, 2E1, 3A4/5 and 2J2 (Samer et al., 2013).

The CYPs that display polymorphism are quantitatively the most important Phase I drug transformation enzymes in mammals. In summary, CYP450 polymorphisms are genetic variations in oxidative drug metabolism characterized by three phenotypes; the poor metabolizer (PM), the intermediate metabolizer (IM), the extensive metabolizer (EM); and the ultrarapid metabolizer (UM). Dramatically reduced or deficient enzyme activity results in the PM phenotype and individuals with PM phenotypes are at risk for elevated concentrations of drugs when administered as the active form, or reduced concentrations when administered as the pro-drug if it is primarily metabolized by the affected enzyme. In such individuals, conventional doses of the drug leading to toxic side effects can be ineffective (pro-drug).

While these tests can be a good predictor of metabolism for some CYP450 enzymes (e.g. CYP2D6), they perform rather poorly for others (e.g. CYP3A4), since regulation of the metabolic phenotype of these enzymes is multifactorial (Zanger and Schwab, 2013). Together with the described monogenetic factors other variables including multi- and epi-genetic factors, sex, age, inflammation, hormonal signalling, diet, co-administration of drugs can play important roles in defining the drug metabolic phenotype of different subjects.

Several other tests have been developed for measuring the metabolic phenotype of CYP450 enzymes (Samer et al., 2013). Debrisoquine and sparteine have been applied for the phenotyping of CYP2D6, but their use is limited by mixed results regarding their safety (Tanaka et al., 2003) and dextromethorphan has been considered the probe of reference for assessment of CYP2D6 activity (Sachse et al., 1997). Similarly, omeprazole serves the phenotyping of CYP2C19 (Chang et al., 1995), while midazolam has been applied, with mixed results, to phenotyping of CYP3A4-5 (Lin et al., 2001) and caffeine is used to probe metabolic activity of CYP1A2 (Ou-Yang et al., 2000). While these tests offer a better readout of CYP450 metabolic phenotype compared to genetic tests, they present several drawbacks. Administration of drugs at therapeutic doses has raised concerns about their safety as diagnostic probes for assessing potential toxicity of drugs under investigation metabolised by the very same enzyme. Furthermore, drug-drug interactions between probes and other concomitant drugs can produce potential side effects (Samer et al., 2013). Moreover, to assess metabolism of these probes, blood samples are collected and metabolic products of the probes are measured. This process is rather lengthy since multiple blood and urine samples have to be collected during a period of at least 8 hours, thus complicating the phenotyping procedure to the level where it is not part of routine care (Samer et al., 2013).

In addition to the blood- and urine-based phenotyping tests, breath tests have been developed to assess metabolism of different drugs through several CYP450 enzymes. 13C-pantoprazole and 13C-methacetine have been applied for the assessment of CYP2C19 activity (Pijls et al., 2014; Thacker et al., 2012), while 13C-dextromethorphan, 13C-caffeine, 13C-erythromycin have been applied for phenotyping of CYP2D6, CYP1A2 and CYP3A4, respectively (De Kesel et al., 2016). These breath tests are based on administration of a 13C-labelled form of the drug or compound under investigation, followed by detection of exhaled 13CO2 in breath (which specifically derives from the administered labelled drug via metabolism through one or few CYP450 enzymes). The clear advantage of breath tests for metabolic phenotyping, compared to blood- or urine-based tests, resides in the non-invasiveness of the sample collection procedure, together with the fact that only one sample collection is usually required. Yet, they are affected by the same drawback as the other phenotyping approaches based on administration of a potentially toxic drug at therapeutic doses, i.e. eliciting potential adverse reactions to the drug probe.

Together with their role in metabolism of multiple drugs, CYP450 enzymes are involved in the biotransformation of a myriad of other xenobiotics. Among these, several molecules contained in foods and drinks, as well as herbal medicaments and other natural products, are processed by CYP450 family. The majority of such compounds have been included in lists approving them for administration to humans such as the generally recognised as safe (GRAS) list generated by the food and drug administration (FDA). When a compound is on such a list they are known to be non-toxic and safe for human consumption. These compounds include terpenes (e.g. limonene and pinene), alcohols (e.g. eucalyptol), amino acids (e.g. glutamic acid), proteins (e.g. casein), elemental compounds (e.g. iron), and vitamins (e.g. ascorbate), among others. Similarly to drugs that are metabolised by one or few CYP450 enzymes, GRAS compounds display enzyme-specificity and are generally biotransformed via specific members of the CYP450 family.

The invention is aimed at addressing the drawbacks of existing methods for assessing the metabolic phenotype of a subject and at providing a test to define a subject's phenotype for metabolizing capacity of liver enzymes to enable prediction of drug efficacy, diagnosis or selection of a treatment regimen and/or toxicity of a xenobiotic. For example, this could help avoid potential drug related toxicity in poor metabolizers and increase efficacy of a treatment by allowing individualisation of therapy in a personalised medicine approach through determination of individual optimized drug selection and dosages.

### SUMMARY

The inventors have found that monitoring or determining the efficiency of biotransformation of a test substance, for example a GRAS compound, by monitoring an exhaled volatile organic compound (VOC) can be applied as a readout of the metabolic activity of a range of metabolising liver enzymes, for example CYP450 enzymes. Alterations of the metabolic phenotype of liver enzymes, as determined by genetic and other concomitant factors, result in alteration of (the rate of) biotransformation of the test substance. This offers the unique possibility of applying, for example, a natural compound as a test substance for determining the metabolic phenotype of one or more liver enzymes non-invasively with a high degree of safety by using a breath test.

The test compound is preferably an exogenous substance. This has advantages over using endogenous compounds as biomarkers in breath as it allows the provision of the test substance at a defined concentration and measurement of the test substance and/or metabolite in breath. The methods described herein thus make use of an exogenous substrate that, when metabolised by the enzyme, offers a readout of enzyme activity. They are based on the use of exogenous volatile organic compound (EVOC) probes as tracers of specific in vivo metabolic activities. EVOC probes as used herein can be volatile compounds that, when administered to a subject through various routes, undergo metabolism and distribution in the body and are excreted via breath. Additionally or alternatively, metabolism of EVOC probes by specific enzymes can lead to production of other volatile compounds (metabolites) that can be detected in breath.

Thus, in one aspect, the invention relates to a diagnostic, non-invasive, in vitro breath test to evaluate metabolism in the liver of a xenobiotic, for example of a therapeutic compound; determine a liver enzyme phenotype, assess a liver disease status or progression. The test includes a method that provides a phenotypic metabolic signature which can be used in a personalised medicine approach for drug administration and/or to detect or predict toxicity of other xenobiotics. The present invention therefore includes a non-invasive breath test which can be used to determine the characteristic of metabolism of a xenobiotic, for example a therapeutic compound, in a subject. This test utilises a test substance which is used as a proxy for a xenobiotic which is metabolised by a liver enzyme.

The term “characteristic of metabolism” includes whether such metabolism occurs, the rate of metabolism and the extent of metabolism. For example, it includes metabolic activity or capacity of a metabolic liver enzyme, for example a CYP450 enzyme, that is the metabolic phenotype of an individual subject. The methods of the present invention utilize the liver enzyme-substrate interaction as they comprise determining the reduction of the concentration of a VOC substrate and/or an increase in the concentration of a VOC metabolite in exhaled breath of a subject. The test substrate thereby acts as a proxy or substitute for a xenobiotic. The subsequent quantification of the substrate and/or metabolite in exhaled breath allows for the determination of pharmacokinetics of the substrate and thus the evaluation of liver enzyme activity (i.e., related metabolic activity/capacity; the metabolic phenotype of a liver enzyme or phenotypic liver enzyme signature). This in turn allows for the determination of the phenotype of a subject with respect to the metabolic activity of the subject's enzymes and conclusions can be drawn as to the rate at which a xenobiotic of interest, such as a therapeutic compound, will be metabolised. There is no need to label the test substance to enable it's detection in breath and the method further conveniently allows for testing of multiple substrates and/or metabolites in one or more sample at the same time. Thus, in the methods provided, the substrate is preferably free of isotope labels and/or other labels.

The invention provides a breath test for assessing a metabolic phenotype.

In particular, the invention provides a method for the assessment of the metabolic phenotype, e.g. a CYP enzyme genotype, of a liver enzyme by using a VOC as a substrate for said enzyme, preferably a GRAS compound as described herein, or by using a substrate which is metabolised to a VOC, as a probe (or reactant) of liver enzyme metabolism.

In one aspect, the invention relates to a method for determining the metabolic activity of a liver enzyme comprising measuring the concentration of a substrate for a liver enzyme and/or measuring the concentration of a metabolite of said substrate in exhaled breath of a subject wherein said substrate and/or its metabolite is a VOC and wherein the substrate is a non-isotope labelled GRAS compound, i.e. an exogenous substance.

In one embodiment of the methods above, the substrate is provided together with a GUARD compound as described herein. The substrate and GUARD compound are both optionally provided as a liquid or fast dissolving/release tablet or capsule.

As will be apparent from the below, such methods can be sued in diagnosing disease, determining progression of disease, determining a metabolic phenotype, or determining the therapeutically effective dosage of a drug.

### FIGURES

The invention is further described in the following non-limiting figures.

FIG. 1. Peppermint-related VOCs in breath before and at 30 minute intervals after consumption of a peppermint capsule. Data from a single individual is shown. The compounds tested were (bars for each time point from left to right): □-Pinene, □-Pinene, D-limonene, Eucalyptol, menthol.

FIG. 2. a) Flow diagram of an embodiment of the method measuring limonene or a metabolite thereof b) Flow diagram of an embodiment of the method measuring limonene or a metabolite thereof using an inducer.

FIG. 3. Flow diagram of an embodiment of the method measuring eucalyptol or a metabolite thereof

FIG. 4. Example of potential outcome on breath concentration of perillyl alcohol, upon administration of limonene and metabolism via CYP450 enzymes. Rapid metabolisers (blue dots) will accumulate perillyl alcohol in their breath with fast kinetics, while poor metabolisers (orange dots) will accumulate the metabolic products more slowly.

FIG. 5. Limonene concentration in the exhaled breath of patients prescribed with an atherothrombotic drug that is metabolised by CYP2C19 and CYP2C9 enzymes. Metabolism of the drug through CYP2C19-2C9 can compete with metabolism of limonene through the same enzymes, thus resulting in higher secretion of limonene into breath.

FIG. 6. This shows CYP phenotyping to determine drug suitability and potential therapeutic range in individuals. It shows time concentration profile for a drug following single dose administration and its pharmacokinetic parameters such as Cmax, Tmax, AUC (area under curve), MTC (minimal toxic concentration) and MEC (minimal effective concentration).

FIG. 7. Comparison of the absorption of a GUARD compound and the target compound (i.e. substrate used).

FIG. 8. Using a CYP inhibitor as a drug proxy.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the invention relates to a method for determining the metabolic activity of a liver enzyme comprising measuring the concentration of a substrate for a liver enzyme and/or measuring the concentration of a metabolite of said substrate in exhaled breath of a subject wherein said substrate and/or its metabolite is a VOC.

In another aspect, the invention relates to a method for determining the therapeutically effective dosage or administration regimen of a therapeutic compound for administration to a subject comprising determining the metabolic activity of a liver enzyme comprising measuring the concentration of a substrate for a liver enzyme and/or measuring the concentration of a metabolite of said substrate in exhaled breath of a subject wherein said substrate and/or its metabolite is a VOC. The method may further include employing said metabolic activity to determine the therapeutically effective dosage of said therapeutic compound for said subject.

In another aspect, the invention relates to a method for selecting a class of therapeutic compounds or a therapeutic compound for administration to a subject comprising determining the metabolic activity of a liver enzyme comprising measuring the concentration of a substrate for a liver enzyme and/or measuring the concentration of a metabolite of said substrate in exhaled breath of a subject wherein said substrate and/or its metabolite is a VOC. The method may further include employing said metabolic activity to select a class of therapeutic compounds or a therapeutic compound for administration to said subject.

In another aspect, the invention relates to a method for determining the toxic dosage of a xenobiotic to a subject comprising determining the metabolic activity of a liver enzyme comprising measuring the concentration of a substrate for a liver enzyme and/or measuring the concentration of a metabolite of said substrate in exhaled breath of a subject wherein said substrate and/or its metabolite is a VOC. The method may further include employing said metabolic activity to determine the toxic dosage of said xenobiotic for said subject.

In another aspect, the invention relates to a method for measuring the rate of metabolism of a xenobiotic, for example a therapeutic compound, in a subject comprising determining metabolic activity of a liver enzyme comprising measuring the concentration of a substrate for a liver enzyme and/or measuring the concentration of a metabolite of said substrate in exhaled breath of a subject wherein said substrate and/or its metabolite is a VOC. The method may further include employing said metabolic activity to determine the rate of metabolism of said therapeutic compound for said subject. In one embodiment, the method may further include employing said metabolic activity to determine the presence of intolerance of said xenobiotic for said subject.

Assessing metabolic activity of liver enzymes through a breath test as described herein can be used to make inferences about the physiological state of the body or health of individual. Such a test can be used for diagnosis of disease, predicting/monitoring progression of disease and/or determining treatment of a disease.

Thus, in another aspect, the invention relates to a method for diagnosing, treating or determining treatment of a metabolic disorder or a disorder associated with a liver enzyme, comprising determining the metabolic activity of a liver enzyme comprising measuring the concentration of a substrate for a liver enzyme and/or measuring the concentration of a metabolite of said substrate in exhaled breath of a subject wherein said substrate and/or its metabolite is a VOC. The method may further include the step of diagnosing or predicting changes in liver metabolic capacity. The method may further include selecting a treatment for said disorder. The method may further include administering said treatment to said subject. Thus, we also provide a method for determining whether a subject suffers from a disorder associated with a liver disease comprising a method having the features as described herein.

In another aspect, the invention relates to a method for determining whether the subject suffers from a disease associated with a liver enzyme, or a disorder associated with a liver enzyme or a disorder impacting liver enzyme-related metabolism, comprising determining metabolic activity of a liver enzyme comprising measuring the concentration of a substrate for a liver enzyme and/or measuring the concentration of a metabolite of said substrate in exhaled breath of a subject wherein said substrate and/or its metabolite is a VOC. The method may further include the step of diagnosing or predicting changes in liver metabolic capacity. The method may further include selecting a treatment for said disorder. The method may further include administering said treatment to said subject. The disorder may be a metabolic disorder.

In another aspect, the invention relates to a method for selecting a subject for a clinical trial to determine the efficacy of a therapeutic compound to treat a disease as the assessment of the metabolic capacity of a liver enzyme can aid in selecting subjects which are most likely to benefit from experimental treatments under investigation in a clinical trial. Thus, the invention relates to a method for selecting a subject for a clinical trial comprising determining metabolic activity of a liver enzyme comprising measuring the concentration of a substrate for a liver enzyme and/or measuring the concentration of a metabolite of said substrate in exhaled breath of a subject wherein said substrate and/or its metabolite is a VOC.

In another aspect, the invention relates to a system for determining metabolic activity of a liver enzyme comprising measuring the concentration of a substrate for a liver enzyme and/or measuring the concentration of a metabolite of said substrate in exhaled breath of a subject wherein said substrate and/or its metabolite is a VOC said system comprising a device for capturing a breath sample from a patient.

In another aspect, the invention relates to a kit comprising a system for determining metabolic activity of a liver enzyme comprising measuring the concentration of a substrate for a liver enzyme and/or measuring the concentration of a metabolite of said substrate in exhaled breath of a subject wherein said substrate and/or its metabolite is a VOC said system comprising a device for capturing a breath sample from a patient. In one embodiment, the kit includes a composition comprising the substrate, such as a GRAS substance. The kit or composition of the kit optionally includes a GUARD compound as described herein.

In another aspect, the invention relates to a method for determining a liver enzyme phenotype, e.g. CYP enzyme genotype, of a subject comprising measuring the concentration of a substrate for a liver enzyme and/or measuring the concentration of a metabolite of said substrate in exhaled breath of a subject wherein said substrate and/or its metabolite is a VOC. In this way, it is possible to determine whether a subject is a poor metabolizer (PM), an intermediate metabolizer (IM), an extensive metabolizer (EM) or an ultrarapid metabolizer (UM) or where a subject sits on spectrum ranging from no metabolism to ultrarapid metabolism.

In another aspect, the invention relates to a method for monitoring the progression of a liver disease in a subject diagnosed with a disease comprising measuring the concentration of a substrate for a liver enzyme and/or measuring the concentration of a metabolite of said substrate in exhaled breath of a subject wherein said substrate and/or its metabolite is a VOC.

According to the various aspects of the invention as described above, the substrate is the test substance which can be used a proxy or substitute for the xenobiotic of interest. As used herein, a “substrate” refers to a chemical compound that is recognized by an enzyme, in this case a liver enzyme, and for which the enzyme catalyzes conversion of the substrate into a different chemical compound which is referred to herein as a “metabolite.” For example, the liver contains enzymes that convert various drug substances (i.e. substrates) to metabolites, which are eliminated from the body in urine, breath or excrement. This enzyme conversion process often determines the duration of action or intensity of drugs, which is why some drugs may be taken several times each day to treat diseases and produce desirable pharmacological effects. The subject may be exposed to the substrate, for example the substrate may be ingested by said subject. In some embodiments, the methods of the invention may include providing or administering a substrate to the subject.

The term xenobiotic refers to a substance that is foreign to the subject's body and which is metabolised by a liver enzyme. In one embodiment, the xenobiotic may a therapeutically effective compound. As explained herein, if the substrate used in the breath test is a proxy for a therapeutic compound or drug, the methods of the invention can be used to determine the dosage of the therapeutic compound based on the subject's metabolic signature. The therapeutic compound may be selected from the non-limiting list including cyclosporin A, tacrolimus, antibiotics such as macrolide antibiotics e.g. erythromycin, anticancer drugs including taxol, small molecule drugs such as ifosfamide, tamoxifen, benzodiazepines, statins, antidepressants, opioids, anti-pscyhotics, such as carbemazepine, anti-coagulants such as warfarine, analgesics, beta-blockers and steroids including testosterone, progesterone, androstenedione and cortisol.

Results of the test can also be used to assess the sensitivity of an individual to one or more environmental pollutants and or toxins to provide insight into current and future health risks associated with that exposure. As an example, this includes the ability to predict the rate by which benzene is broken down by CYP2E1 into its toxic metabolites. Similarly, results of the test may be used to assess damage of environmental exposures to liver enzymes such as the destruction of CYP2B1 by inhalation of benzene.

Thus, in another embodiment, the xenobiotic may be a noxious or toxic substance or a substance that is metabolised into a toxic substance, for example an environmental pollutant. In one example, the noxious substance is a hydrocarbon, such as benzene or a derivative thereof, for example methylbenzene (Toluol). As explained herein, if the substrate is a proxy for a noxious substance, the methods of the invention can be used to determine the toxicity of the substance on said individual or the tolerance of said individual to said noxious substance based on the subject's metabolic signature.

The term VOC refers to any compound of carbon, excluding carbon monoxide, carbon dioxide, carbonic acid, metallic carbides or carbonates and ammonium carbonate, which participates in atmospheric photochemical reactions. Thus, the VOC is not carbon dioxide or carbon monoxide. Generally, VOCs are defined as organic chemical compounds whose composition makes it possible for them to evaporate under normal indoor atmospheric conditions of temperature and pressure. Since the volatility of a compound is generally higher the lower its boiling point temperature, the volatility of organic compounds is sometimes defined and classified by their boiling points. In one embodiment, a VOC is any organic compound having an initial boiling point less than or equal to about 250° C. measured at a standard atmospheric pressure of about 101.3 kPa. The VOC that is measured according to the methods is not endogenous to the subject. This ensures that any readings are not contaminated by endogenous VOC that are naturally produced.

In the methods, kit and system as provided and explained above, the test substance (also termed target substance or substrate) is preferably an exogenous compound that does not naturally occur in the test subject and that is recognized by an enzyme that occurs naturally in the body of a subject and for which the enzyme catalyzes conversion of the substrate into a different chemical compound. The latter is referred to herein as a “metabolite”. The test substance is also non-therapeutic. The substrate is absorbed into the blood.

The exogenous substrate is used as a probe for enzymatic activity by monitoring the breath clearance (or washout) in a subject's breath of the substrate itself, and/or by detecting a metabolic product derived from metabolism of the substrate. Thus, the substrate is not a drug, i.e. it does not have any therapeutic benefit. In one embodiment, the test substance, i.e. the substrate, is a GRAS compound. “GRAS” is an acronym for the phrase Generally Recognized As Safe. Under sections 201(s) and 409 of the Federal Food, Drug, and Cosmetic Act, any substance that is intentionally added to food is a food additive, that is subject to premarket review and approval by FDA, unless the substance is generally recognized, among qualified experts, as having been adequately shown to be safe under the conditions of its intended use, or unless the use of the substance is otherwise excepted from the definition of a food additive. For example, the GRAS compound can be a naturally occurring compound. For example, the GRAS compound can be selected from a food or food additive. In one embodiment, the GRAS compound is a vitamin, phenolic flavoring agent, natural oil, alcohol, amino acid or antioxidant. In one embodiment, the GRAS compound is a plant extract. In one embodiment, the GRAS compound is a plant substance primarily used for flavoring, coloring or preserving food. In one embodiment, the GRAS compound is an aliphatic or aromatic terpene hydrocarbon or a terpenoid. In one embodiment, the GRAS compound is limonene or eucalyptol. In one embodiment, the metabolite is a perillyl alcohol.

In the methods described herein, the substrate is provided at a pre-determined quantity.

Preferably, one embodiment of the various aspects of the invention, the substrate is not labelled, for example not isotope labelled. Thus, the substrate is not labelled with an isotope, such as, for example, 12C, 13C, 14C, 2H, 14N or 18O.

According to the various aspects of the invention, in one embodiment, the substrate is a VOC, e.g. non-isotope labelled GRAS compound,and the concentration of the exhaled VOC substrate in breath is measured. In another embodiment, the substrate is not a VOC, but is converted by enzymatic action of a liver enzyme into a metabolite that is a VOC and the concentration of the exhaled VOC metabolite in breath is measured. In another embodiment, the substrate and the metabolite are both a VOC and the concentration of the exhaled VOC substrate and/or VOC metabolite in breath is measured. The kinetics of metabolism and subsequent breath excretion of the exogenous VOC probe, or of its products, can be used as an indication of the metabolic activity of specific enzymes or organs/tissues. In case breath levels of the exogenous VOC probe itself are to be monitored, clearance or washout of the exogenous VOC probe will be a function of the metabolic activity of the enzyme(s) under investigation. On the contrary, if breath secretion of the product(s) originating from the exogenous VOC probe are to be determined, the rate of product generation will be associated to the enzymatic activity of interest.

As used herein, “healthy subject” is defined as a subject that does not have the disease of interest.

As used herein, “reference value” means a value determined by performing the testing method on a plurality of reference subjects. A reference subject can be a healthy subject or a subject diagnosed with a disease.

A “likelihood of a disease state” means that the probability that the disease state exists in the subject specimen is about 50% or more, for example 60%, 70%, 80% or 90%.

The term liver disease, or metabolic disease as mention herein refers to metabolic condition, i.e. metabolic liver disease, that is characterised by differential expression or activity of a liver enzyme. In one embodiment, this is associated with a liver enzyme, e.g. a CYP enzyme, e.g. differential expression or activity of a CYP enzyme. In one embodiment, this is selected from non-alcoholic fatty liver disease (NAFLD), NASH, liver failure, damage to the liver, cirrhosis. In one embodiment, the liver disease is not associated with alcohol abuse.

According to the aspects of the invention, the concentration of the substrate and/or metabolite can be used to determine the rate of metabolism of the substrate and/or metabolite as follows. The rate of metabolism of the substrate and/or metabolite can be calculated from the initial dose of the substrate and/or metabolite, the amount of time elapsed between the time the initial dose is given and the time the exhaled breath of the subject is analysed for the substrate and/or metabolite, and the concentration of the substrate and/or metabolite. For the collection of a breath sample and methods of measurement, the device and methods described in WO2017/187120 or WO2017/187141 (both publications are hereby incorporated by reference) can be used.

Metabolism and transformation of the substrate by one or more enzyme leads to the generation of a metabolic product of that enzymatic reaction, i.e. a metabolite. Soon after provision of the substrate, the substrate is excreted into breath at high levels and clearance of the substrate from breath occurs as a consequence of biotransformation of the substrate by the action of one or more disease-specific enzymes (washout of the reactant). For example, the kinetic profile of the clearance of the substrate from breath is used as the readout of enzyme activity responsible for biotransformation of said substrate.

In addition, metabolism of a specific substrate through one or more enzyme leads to production of enzyme-specific metabolic products. As opposed to washout curves of the substrate, metabolic products are excreted into breath over time, starting at low levels and increasing over time due to biotransformation of the substrate by the disease-specific enzyme. Measurement of such a metabolic product is applied as a probe for assessing the metabolic phenotype of the enzyme or enzymes responsible for the production of said product. A substrate and corresponding metabolic product(s) can thus be used either alone or in combination to assess the activity of one or more disease-specific enzyme.

Therefore, the methods provided herein enable the testing of multiple compounds in exhaled breath. This allows testing for the presence of more than one type of disease. Furthermore, multiple compounds which are specific to a certain type of disease can be measured in breath thereby enabling a more accurate diagnosis due to multiple parameters that are assessed. In one embodiment, the invention therefore relates to a method for the detection of a disease comprising assessing the activity of one or more disease-specific enzyme by measuring the concentration of two or more exogenous substrates for said enzyme and/or measuring the concentration of two or more metabolites of said substrate(s) in exhaled breath of a subject.

In one embodiment, the methods for the detection of a liver disease disclosed herein comprise assessing the activity of more than one enzyme by measuring the concentration of two or more exogenous substrates for said enzyme and/or measuring the concentration of two or more metabolites of said substrate(s) in exhaled breath of a subject. The method described herein can therefore be a multiplex method enabling assessment of multiple enzymatic activities simultaneously in the same breath sample(s).

In one embodiment, the methods of the invention comprise collecting a breath sample. The breath sample can include air exhaled from one or more different parts of the subject's body (e.g. nostrils, pharynx, trachea, bronchioles, alveoli etc). In one embodiment, the fraction of breath most correlated with the concentration of the test compound in blood is captured.

By monitoring pressure and CO2 and capturing the fraction of breath dominated by alveola air it is possible to collect the fraction of breath most correlated with the concentration in blood. This aids in correcting for variation in how a person breaths and physiological differences between people such anatomical dead space.

Breath samplers such as a ReCIVA breath sampler can be used to sample air (see US20170303823A1 and US20170303822A1).

Chemicals such as limonene are prevalent in air and originate from many sources such as cleaning products and food. The effect of such chemicals on measurements can be mitigated by the use of a clean air source, such as a CASPER clean air source, in particular when the patient inhales air that is cleaned by the clean air source, such as a CASPER clean air source, and then exhales into the breath sampler, such as a ReCIVA, for subsequent collection Thus, in one embodiment, the sample is exhaled breath wherein the subject inhaled air that is cleaned by the clean air source.

In one embodiment, the methods disclosed herein include measuring a control. In particular, the methods disclosed herein include measuring the concentration of a second compound that has substantially the same absorption characteristics as the substrate, i.e. absorption into the blood. Thus, this compound is provided to the test subject together with a substrate. The second compound is termed GUARD compound herein. This is an exogenous (i.e. a substance that does not naturally occur in the subject), non-toxic, non-therapeutic substance. It is preferably a GRAS compound and is an exogenous substance.

The GUARD compound and/or its metabolite is a VOC and can thus be measured in breath. It is not isotope-labelled and optionally does not have any other labels. It is absorbed into the blood.

The GUARD compound is provided at a pre-determined concentration.

It is advantageous to use the GUARD compound as a control in the methods to obtain a better read out and reduce background noise. The GUARD compound is a secondary compound with substantially the same absorption characteristics as the substrate, but it is eliminated from blood with a longer time constant. By substantially it is meant that the absorption characteristics are identical to the absorption characteristics of the substrate or nearly identical, that is vary by up to 10% or up to 5%., e.g. 0.5%, 1%, 2%, 3% 4%, 5% The skilled person will appreciate that numerous GRAS compounds can be used. One non limiting example is an organosulfur compound, such as diallyl sulphide.

By including a step in the methods that measures the ratio of this compound to the target (i.e. substrate), systematic variations can be corrected for such as shallow breathing in one collect versus another, if the absorption characteristics are well matched then variations in absorption can also be corrected for. Thus, the various methods described herein also include measuring the concentration of a GUARD compound.

The concentration of the substrate, metabolite or GUARD compound can be measured using methods known in the art. The concentration as used herein means the content or mass of the substrate and/or metabolite in exhaled breath as expressed, for example in grams/litre (g/l). In one embodiment, concentration is measured over time, for example by measuring the kinetics of the clearance. For example, concentration is measured by assessing the kinetic profile of the clearance of the substrate or GUARD compound from breath which is then used as a readout. In addition or alternatively, secretion of metabolic products that can derive from the substrate or GUARD compound can be measured over time. For example, clearance of the substrate from breath and secretion of metabolic products can both be measured in the same breath sample at the same time or at different times.

In another aspect, we also provide the use of GUARD compound as described above as a control in a breath test, such as a breath test for analysing the metabolic activity of a liver enzyme in which the test compound is an exogenous GRAS compound.

In one embodiment, the concentration or amount of the substrate and/or its metabolite may be determined in absolute or relative terms in multiple breath samples, e.g. in a first breath sample (collected at a first time period) and in a second breath sample (collected at a later, second time period), thus permitting analysis of the kinetics or rate of change of concentration thereof over time. Thus in one embodiment, the method comprises collecting different selected exhaled breath samples, or fractions thereof, on a single breath sample capture device, the method comprising the steps of:

(a) collecting a first exhaled breath sample by contacting the sample with a capture device comprising an adsorbent material;

(b) collecting a second exhaled breath sample by contacting the second sample with said capture device, wherein the first and second exhaled breath samples are caused to be captured on the capture device in a spatially separated manner.

In one embodiment, when an appropriate reference is indicative of a subject being free of a liver disease, a detectable difference (e.g., a statistically significant difference) between the value determined from a subject in need of characterization or diagnosis of a disease and the appropriate reference may be indicative of the disease in the subject. In one embodiment, when an appropriate reference is indicative of the disease, a lack of a detectable difference (e.g., lack of a statistically significant difference) between the value determined from a subject in need of characterization or diagnosis of a disease and the appropriate reference may be indicative of the disease in the subject.

Thus, in one aspect, the methods include detecting the concentration of the substrate and/or metabolite in exhaled breath from the subject, and diagnosing the subject as having a likelihood of a liver disease state if the level of one or more of the substrate and/or metabolite is different from the healthy subject value.

The methods of the invention may further include the step of measuring the concentration of the substrate and/or metabolite in response to a modulator of a liver enzyme. The modulator of a liver enzyme may be a therapeutic drug that is metabolised by a liver enzyme, e.g. an anticancer drug. This can help develop personalised treatment plans as the metabolic phenotype of the subject will inform on the drug dosage.

The methods of the invention may further include the step of selecting a treatment for said disease. The methods may further include administering said treatment to said subject.

In some embodiments the capture device comprises an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. In one embodiment, the material is Tenax GR, which comprises a mixture of Tenax® TA and 30% graphite. One Carbograph® adsorbent is Carbograph 5TD. In one embodiment, the capture device comprises both Tenax GR and Carbograph 5TD. The capture device is conveniently a sorbent tube. These are hollow metal cylinders, typically of standard dimensions (3½ inches in length with a ¼ inch internal diameter) packed with a suitable adsorbent material.

In one embodiment, metabolic activity of one or more liver enzyme of a subject is determined from the measured concentration and compared with one or more reference value. The reference value may be that of a standard population. A standard population may be a healthy untreated population. Alternatively, a standard population may be a population of individuals treated with a specific therapeutic compound that is metabolised by a liver enzyme. The reference value may also be that of a subject with a known compromised liver enzyme metabolism.

This comparison with one or more reference value provides the metabolic phenotype of the subject and allows determining whether the subject is a poor metabolizer (PM), an intermediate metabolizer (IM), an extensive metabolizer (EM) or an ultrarapid metabolizer (UM) or where a subject sits on spectrum ranging from no metabolism to ultrarapid metabolism. Such information can then, for example, be used to determine the proper dosing regimen of a therapeutic compound for the subject using pharmacokinetic equations or to determine sensitivity of a subject to potentially noxious xenobiotics.

A liver enzyme as used herein is an enzyme that is expressed in the liver. The enzyme may be expressed exclusively in the liver or it may be expressed predominantly in the liver, but may also be expressed in other tissues in the body of a subject. It is, for example, involved in the metabolism of lipophilic xenobiotics including therapeutic drugs, chemical carcinogens and environmental toxins.

In one embodiment of the various aspects of the invention, the liver enzyme is a CYP450 enzyme. CYP450s are a large family of heme-containing enzymes that, in addition to the endogenous role in cell proliferation and development, includes many catalysts for detoxification and activation of lipophilic xenobiotics including therapeutic drugs, chemical carcinogens and environmental toxins. All mammals share at least 14 CYP450 families but most drug metabolism is catalyzed by only three families: CYP1, CYP2 and CYP3. In one embodiment, the CYP450 enzyme is selected from families 1, 2 or 3. For example, the CYP450 enzyme is selected from CYP1A1, CYP1A2, CYP1B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7 or CYP3A43. In one embodiment, the enzyme is CYP2C19, CYP2C9 and/or CYP3A4.

In another embodiment, the liver enzyme is selected from glutathione S-transferase, aryl sulfatase and UDP-glucuronyl transferase or aldehyde dehydrogenases.

In one embodiment, the liver enzyme is CYP2C19 and/or CYP2C9 and the substrate is limonene. In one embodiment, the liver enzyme is CYP2C19 and/or CYP2C9 and the substrate is limonene and the metabolite is a perillyl alcohol. In one embodiment, the liver enzyme is CYP3A4 and the substrate is eucalyptol.

A metabolic disorder as used herein refers to a disorder where the body's usual metabolic processes are disrupted. This includes damage to or disease of the liver, including progression from NAFLD, to NASH, to Cirrhosis, and liver failure.

As explained above, the methods can be used to determine whether someone is an ultra-rapid, rapid, intermediate or poor metabolizer for a specific enzyme. This helps to identify whether someone is likely to have a beneficial, ineffective or toxic response to a therapeutic compound. In general, this allows selection of a suitable therapy and/or suitable dosage of the therapeutic compound in a personalized medicine approach. This includes the following examples:

CYP2C19

Utilizing evaluation of CYP2C19 to predict the effectiveness of Clopidogrel (Plavix) for secondary prevention of atherothrombotic events after an ischemic event such as a myocardial infarction, ischemic cerebral event, peripheral arterial conditions or acute coronary syndrome.

Determining effectiveness of CYP2C19 can be used to assess the effectiveness of proton pump inhibitors such as omeprazole, lansoprazole and pantoprazole. This can be used to predict effectiveness of Helicobacter pylori eradication therapy allowing selection of appropriate therapy, gastroesophageal reflux, NSAID-induced GI-tract damage and healing of gastric ulcers.

Similarly, CYP2C19 is relevant to determine the effectiveness and potential toxicity of antidepressant drugs (SSRI's/TCA's) such as citalopram, amitriptyline, moclobemide.

Additionally, CYP2C19 can be used to assess toxicity, addictiveness and effectiveness of benzodiazepines such as clobazam and diazepam.

Through its effect on proguanil the effectiveness of anti-malarial therapy can be predicted and monitored.

CYP2C19 enzymatic activity prediction can also be used to predict the response of postmenopausal woman with breast cancer to tamoxifen enabling selection of appropriate therapeutic strategies.

CYP2C9

Evaluation of the metabolic activity of CYP2C9 can be used to assess toxicity and effectiveness of warfarin and it's interaction with other drugs such as simvastatin metronidazole or macrolid antibiotics. This can help prevent complications such as bleeding whilst potentially negating the need for frequent INR checks and dose adjustments.

Furthermore, assessment of CYP2C9 metabolism can be used to predict effectiveness and toxicity for patients with diabetes mellitus type 2 using first or second generation sulfonylurea hypoglycaemic drugs such as glibenclamide, tolbutamide and glimepiride. This helps keeping blood glucose levels in check and prevents hypoglycaemic events.

Similarly, assessment of CYP2C9 metabolism can be used to assess effectiveness and toxicity of anticonvulsants such as valproic acid and phenytoin in patients with epilepsia, manic depression or migraine.

Angiotensin receptor blockers candesartan and losartan are furthermore metabolized by CYP2C9. A test assessing CYP2C9 metabolic effectiveness can aid in assuring treatment for hypertension is effective.

Most NSAIDs are metabolized by CYP2C9. A test such as the one described can be used to predict and prevent gastrointestinal bleeding as a consequence of chronic NSAID use.

CYP3A4

Assessing CYP3A4 metabolic function can be used to determine what dosage of statin a patient needs to receive to achieve effective lowering of cholesterol levels. These include drugs such as simvastatin, lovastatin and atorvastatin.

Furthermore, assessment of CYP3A4 metabolism can be used to titrate the dosage of tacrolimus in patients after a renal transplant to help prevent rejection of the graft.

CYP3A4 metabolises a lot of steroids such as Dexamethasone and methylprednisolone and can be used to predict and prevent toxicity such as Cushings.

Furthermore, CYP3A4 can help assess the right type and dosage of antidepressants such as SSRI's and TCA's through its metabolism of drugs like amitriptyline, imipramine, citalopram, norfluoxetine and sertraline.

Examples as stated above indicate the test can also be used to predict drug-drug interactions for novel and existing therapeutics by evaluating the activity of one or more drug metabolizing liver enzyme with (a combination of) breath test(s).

In one aspect, the method for determining metabolic activity can be used as a multiplex method to measure multiple substrates and/or multiple metabolites. Accordingly, in one embodiment, the concentration of two or more metabolites of a single substrate for a liver enzyme, such as a CYP450 enzyme, is measured.

In another embodiment, the concentration of two or more metabolites of two or more substrates for a liver enzyme is measured. In another embodiment, the concentration of two or more substrates for a liver enzyme is measured.

In one embodiment, the methods provided further includes the step of measuring the concentration of the substrate or a metabolite of the substrate in a blood, urine or saliva sample.

In another embodiment, the methods provided further includes the step of determining the CYP450 genotype of a subject. This can be done using genetic tests known in the art.

In another embodiment, the methods provided further includes the step of measuring the concentration of the substrate or metabolite in response to a liver enzyme modulating agent, such as an inducer, substrate or inhibitor of a liver enzyme. A liver enzyme modulating agent can be any compound that alters (e.g., increases or decreases) the expression level or biological activity level of a liver enzyme polypeptide compared to the expression level or biological activity level of CYP450 polypeptide in the absence of the liver enzyme modulating agent. A liver enzyme modulating agent can be a small molecule, polypeptide, carbohydrate, lipid, nucleotide, or combination thereof. The liver enzyme modulating agent may be an organic compound or an inorganic compound. Examples are shown in the figures.

In one embodiment, the method includes the step of providing the substrate to a subject, for example by various administration routes. Administration may by any convenient route, including but not limited to oral, topical, parenteral, sublingual, rectal, vaginal, ocular, intranasal, pulmonary, intradermal, intravitrial, intramuscular, intraperitoneal, intravenous, subcutaneous, intracerebral, transdermal, transmucosal, by inhalation, or topical, particularly to the ears, nose, eyes, or skin or by inhalation. Parenteral administration includes, for example, intravenous, intramuscular, intraarterial, intraperitoneal, intranasal, rectal, intravesical, intradermal, topical or subcutaneous administration. Preferably, the compositions are administered orally. In one embodiment, administration is sublingually.

The substrate can be contained in a composition, such as a nutritional supplement. The different absorption rates of the substrate into the blood can cause significant shifts in the time of maximum concentration on breath. Therefore, in one embodiment, the substrate is provided in a formulation to ensure fast delivery. In one embodiment, the substrate is formulated as a liquid. In another embodiment, the substrate is formulated as a fast release/fast dissolving tablet or capsule. This ensures that the absorption has a much shorter time constant compared to the washout.

In another embodiment, the subject is fasting overnight and fasting can be combined with the provision of the substrate as a liquid or fast release/dissolving tablet or fast release/dissolving capsule or other oral administration format.

The composition can also include a GUARD compound as described herein, i.e. a non-isotope labelled GRAS compound that is a VOC or where the metabolite is a VOC.

In one embodiment, the various methods disclosed herein comprise the following steps

a) selecting a liver enzyme for analysis;

b) selecting exogenous substrate for said enzyme;

c) providing the substrate to a test subject and

d) measuring the concentration of an exogenous substrate for the enzyme and/or measuring the concentration of a metabolite of said substrate in exhaled breath of a subject wherein the substrate and/or its metabolite is a VOC as described herein.

Further steps include the concurrent provision of the GUARD compound as described herein and measuring the ratio of this compounds to the substrate. This can be provided separately, but at the same time, i.e. in the form of a separate composition. It can also be provided in the same composition, i.e. the substrate and GUARD compound are both part of the same composition.

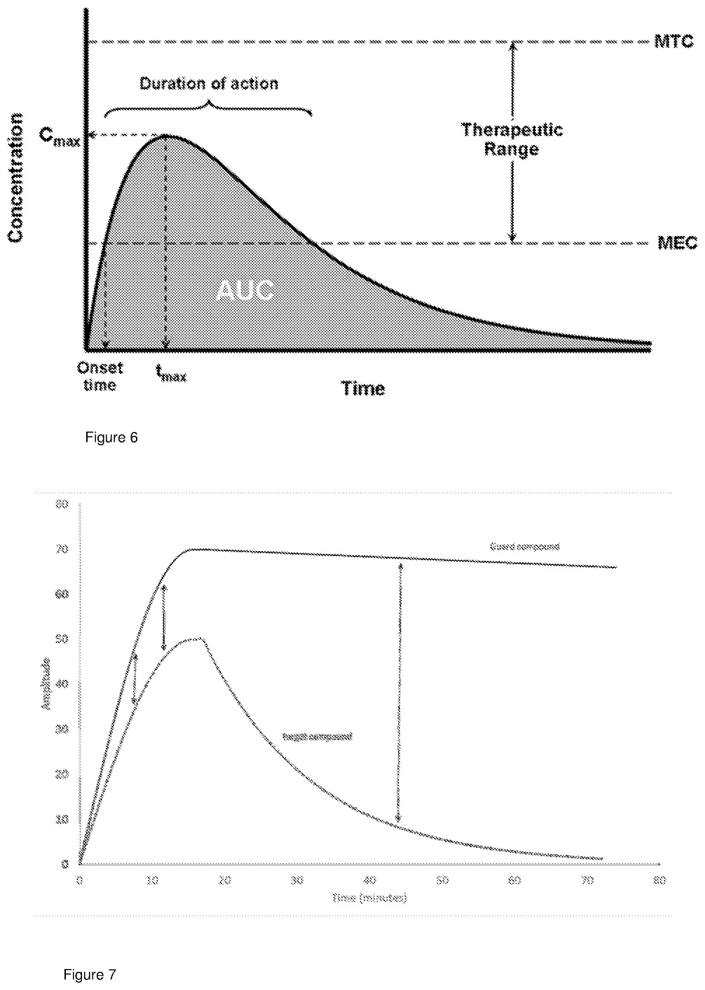

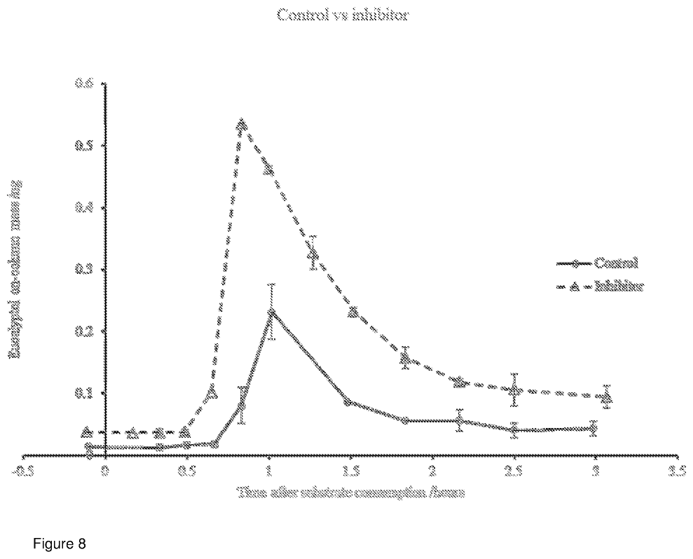

Data analysis can be carried out in the following way:
Primary analysis by differing gradient as elimination is considered 1^{st} order so a simple exponential;
Area under curve can also be calculated (e.g. see FIG. 6);
Ratio to GUARD compound is the most error resistant way to measure a change in activity, it is also possible to start seeing differences in the absorption phase before the peak of the target compound so opens the way to a faster test for the patient;
Measuring concentration at a specific and pre-detemrined single or multiple time point(s).

Monitoring the metabolite rather than the substrate can be carried out in some embodiments as this may provide specificity (such as for limonene where two enzymes act on the substrate, it but produces different metabolites).

The kit as described herein includes a composition. This can be for administration as described above. It may also include a pharmaceutically acceptable carrier or vehicle. This can be a particulate, so that the compositions are, for example, in tablet or powder form. The term “carrier” refers to a diluent, adjuvant or excipient, with which a substrate is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. The composition can be in the form of a liquid, e.g., a solution, emulsion or suspension. The liquid can be useful for delivery by injection, infusion (e.g., IV infusion) or sub-cutaneously. As a solid composition for oral administration, the composition can be formulated into a powder, granule, compressed tablet, pill, capsule, chewing gum, wafer or the like form. Compositions can take the form of one or more dosage units.

The substrate is not provided as part of a drug substance, i.e. it is not an additive in a drug matrix to measure compliance to a treatment schedule.

Typically, the amount of the substrate administered as part of the methods described herein or the amount of the substrate included in the composition comprised in the kit is at least about 0.01% of the substrate by weight of the composition. When intended for oral administration, this amount can be varied to range from about 0.1% to about 80% by weight of the composition. For administration by injection, the composition can comprise from about typically about 0.1 mg/kg to about 250 mg/kg of the subject's body weight, preferably, between about 0.1 mg/kg and about 20 mg/kg of the subject's body weight, and more preferably about 1 mg/kg to about 10 mg/kg of the subject's body weight.

In one embodiment, the system for determining metabolic activity of a liver enzyme includes a device for capturing a breath sample as described in WO2017/187120 or WO2017/187141. The device in WO2017/187120 comprises a mask portion which, in use, is positioned over a subject's mouth and nose, so as to capture breath exhaled from the subject. The exhaled breath samples are fed into tubes containing a sorbent material, to which the compounds of interest adsorb. After sufficient sample has been obtained, the sorbent tubes are removed from the sampling device and the adsorbed compounds desorbed (typically by heating) and subjected to analysis to identify the presence and/or amount of any particular compounds or other substances of interest. The preferred analytic technique is field asymmetric ion mobility spectroscopy (abbreviated as “FAIMS”). The method in WO2017/187141 refinement of the method described in WO2017/187120 is disclosed in WO2017/187141. In that document, it is taught to use breath sampling apparatus substantially of the sort described in WO2017/187120, but in a way such as to selectively sample desired portions of a subject's exhaled breath, the rationale being that certain biomarkers or other analytes of interest are relatively enriched in one or more fractions of the exhaled breath, which fractions themselves are relatively enriched in air exhaled from different parts of the subject's body (e.g. nostrils, pharynx, trachea, bronchioles, alveoli etc.).

In a further aspect, we provide an in vitro method for identifying a VOC for use in a method described herein comprising exposing a liver enzyme or liver disease tissue to a test VOC and measuring the metabolism of the VOC to assess specificity and activity of the enzyme for the test VOC. In one embodiment, the method uses a library approach and an array of compounds is screened. Standard enzyme assays can be used to measure metabolism.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### EXAMPLES

The invention is further described in the following non-limiting examples.

### Example 1

Measuring GRAS VOCs Compounds in Breath

By measuring VOCs in breath following ingestion of a peppermint capsule we have shown that Breath Biopsy® can be used to observe the decrease in target compounds over time using repeated, robust breath collection and analysis over a period of 8 hours.

Breath Biopsy Workflow

After ingestion of the peppermint capsule, breath samples were collected from an individual onto a Breath Biopsy® Cartridge every 30 minutes for 8 hours using a Breath Sampler as described in WO2017/187120. For comparison, two breath collections were made from the same individual prior to ingestion to provide a baseline concentration for the VOCs of interest. Breath samples were analysed in the Breath Biopsy® Clinical Lab by FAIMS and TD-GC-TOF mass spectrometry.

VOCs in Breath Following Capsule Ingestion

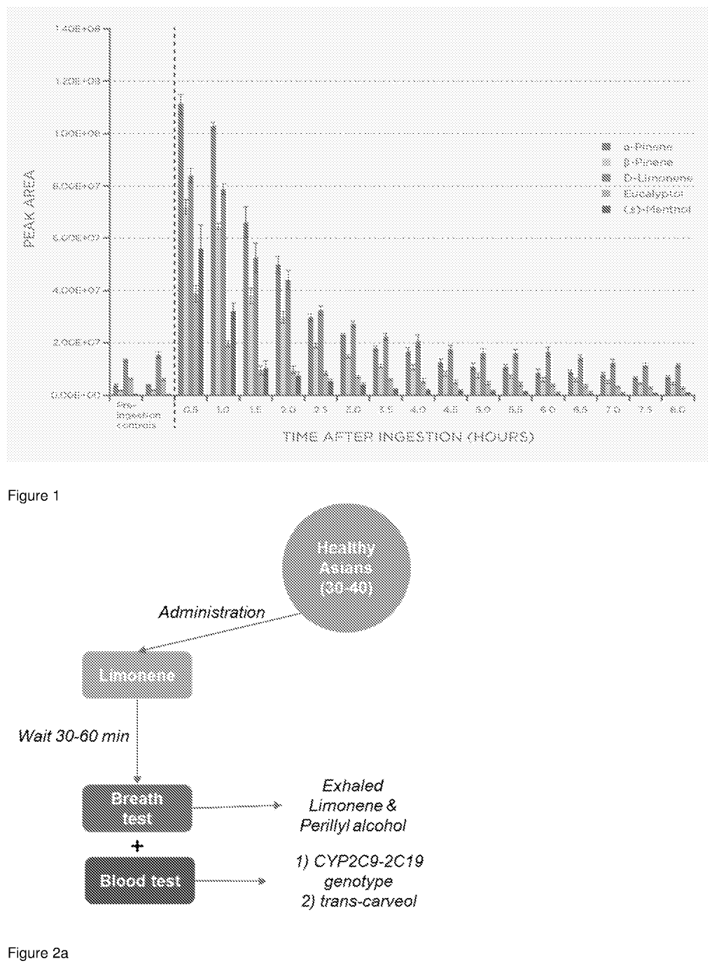

Analysis of breath captured 30 minutes after consumption of the peppermint capsule shows a large increase in the VOCs α-pinene, β-pinene, limonene, eucalyptol and (±)-menthol compared to baseline pre-ingestion controls captured immediately prior to taking the capsule (FIG. 1). The most abundant of these peppermint-related compounds are α-pinene, β-pinene and limonene. Limonene was present at part-per-trillion (ppt) concentrations.

Breath collections made every 30 minutes after this initial capture show a consistent decrease in the target VOCs over time. Captures made from 6.5 hours after consumption show the levels of the target VOCs decreasing to baseline levels. All of the target compounds display a similar washout curve over time.

Longitudinal Monitoring of VOCs in Exhaled Breath

In this study, standard deviations were calculated for the 4 replicate samples collected on the Breath Biopsy® Cartridge at each breath collect (Table 1). This gives an indication of the high intra-sample reproducibility of breath sampling and analysis using the Breath Biopsy® platform.

TABLE 1

| % RSD | α-pinene | β-pinene | limonene | eucalyptol | (±)-menthol |
|---|---|---|---|---|---|
| average | 8.05 | 7.99 | 7.38 | 11.26 | 27.53 |
| min | 1.34 | 1.99 | 2.51 | 4.94 | 4.14 |
| max | 18.60 | 13.02 | 11.60 | 19.46 | 61.83 |

Mean average and range of % relative standard deviation (% RSD) of peak area for compounds shown in Figure 1. Note high max % RSDs are for points close to baseline where VOC concentrations were much lower.

### SUMMARY

This study demonstrates that GRAS VOCs can be used to reproducibly captured and analysed in breath samples. Using a peppermint capsule as a surrogate pharmaceutical, the levels of peppermint-related compounds in breath were found to increase rapidly after ingestion, and subsequently decrease following a washout curve over time.

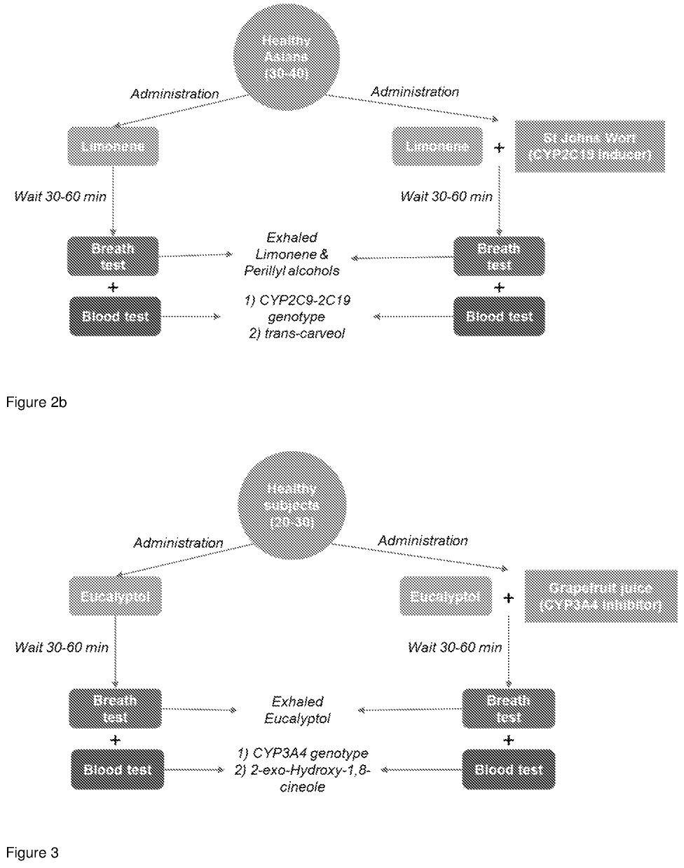

Monitoring the concentration of limonene and/or its metabolite(s) in breath, and its decay over time, fro example after administration of exogenous limonene, can indicate metabolism of limonene through CYP2C9-2C19 and therefore can be used as a proxy for enzymatic activity. This is illustrated schematically in FIG. 2.

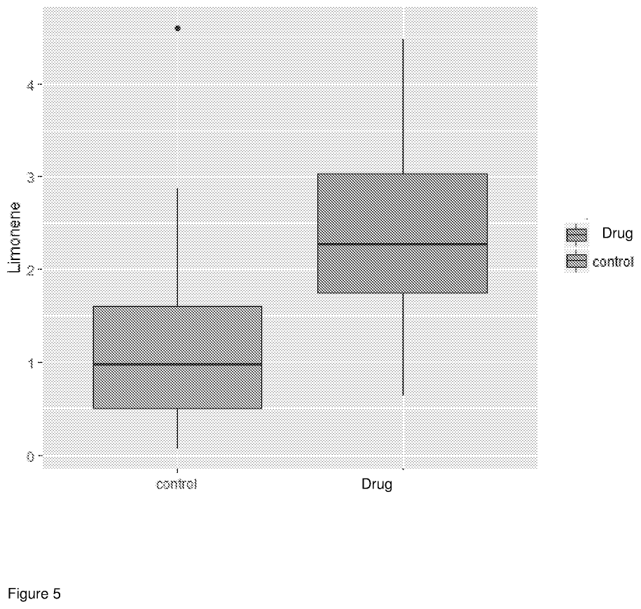

FIG. 5 shows the results of a clinical study comparing the concentration of limonene in exhaled breath in a control group of untreated patients with the concentration of limonene in exhaled breath in patients treated with a drug for an atherothrombotic event. The drug is metabolised by CYP2C19 and CYP2C9 enzymes. Metabolism of the drug through CYP2C19-2C9 can compete with metabolism of limonene through the same enzymes, thus resulting in higher secretion of limonene into breath. As less limonene is metabolised compare dot eh control group. This demonstrates that limonene can be used as a proxy for a therapeutic compound.

### Example 2 Determining CYP450 Enzyme Metabolic Capacity

Soon after administration, the reactant or substrate is excreted into biofluids at high levels and clearance of the reactant from biofluids occurs as a consequence of biotransformation of the reactant by the action of CYP450 enzymes (wash-out curves). As shown above, exhaled reactant can be measured in a subject's breath. The kinetic of clearance of the reactant from biofluids is used as a readout of the metabolic phenotype of the specific CYP450 responsible for biotransformation of said reactant.

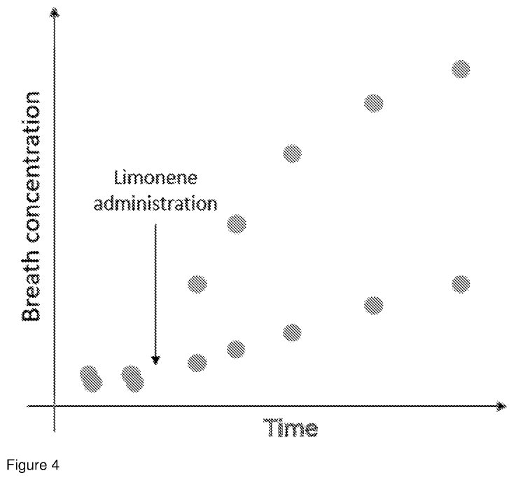

In addition, metabolism of the reactant through specific CYP450 enzymes leads to production of enzyme-specific metabolic products. As opposed to wash-out curves of the reactant, metabolic products are excreted into biofluids over time, starting at low levels and increasing due to biotransformation of the reactant by specific CYP450 enzymes (see FIG. 4). Measurement of such metabolic products is applied as a probe for assessing the metabolic phenotype of the enzyme or enzymes responsible for the production of said product. Reactant and corresponding metabolic product(s) are used in isolation or in combination to assess the metabolic phenotype of the CYP450 enzyme or enzymes responsible for that reaction. These approaches are shown in FIGS. 2 and 3.

The measurements of reactant and/or metabolite in the exhaled breath of a subject can be compared to a reference level of reactant and/or metabolite obtained from a reference population.

### Example 3 Assessment of CYP3A4 Enzymatic Activity Using Eucalyptol as a Substrate

The methods as described herein can be used for the assessment of CYP3A4 enzymatic activity. CYP3A4 is responsible for the metabolism of ˜40% of all prescribed drugs, including immunosuppressants like cyclosporin A and tacrolimus, macrolide antibiotics like erythromycin, and anticancer drugs including taxol, smaller molecules including ifosfamide, tamoxifen, benzodiazepines, several statins, antidepressants, opioids and many more. CYP3A4 is also an efficient steroid hydroxylase with an important role in the catabolism of several endogenous steroids including testosterone, progesterone, androstenedione, cortisol and bile acids.

Eucalyptol is a volatile compound contained in many foods and food supplements and is listed among the GRAS list of compounds. Importantly, eucalyptol is specifically metabolised by CYP3A4 (Miyazawa et al., 2001). Oral administration of eucalyptol leads to accumulation of eucalyptol in the blood stream, followed by secretion in breath. Metabolism of eucalyptol through CYP3A4 decreases blood levels of eucalyptol over time, subsequently reducing the amounts of eucalyptol exhaled in breath. Monitoring the concentration of eucalyptol in breath, and its decay over time after administration of exogenous eucalyptol, can indicate metabolism of eucalyptol through CYP3A4 and therefore can be used as a proxy to assess CYP3A4 enzymatic activity (i.e. CYP3A4 metabolic activity). This is illustrated schematically in FIG. 3. Thus, on the basis of this test, conclusion can be drawn as to the rate at which a subject metabolises a xenobiotic, such as a therapeutic compound.

### Example 4 Study Using a Drug Proxy

Control versus inhibitor experiment in same person, some specific CYP450 enzymes are inhibited by grapefruit juice, in this case CYP3A4 enzyme is being probed with Eucalyptol. This enables a study to be carried out within a single person inducing two distinct enzyme activity levels. Grapefruit juice is thus used as a drug proxy.

For the control arm, the person was asked to:

1. Fast at least 8 hrs prior to giving first sample. Water can be consumed

2. Not brush teeth within 2 hrs of giving first sample

3. Not drink citrus juice, including orange and grapefruit juice or earl grey tea in 3 days before collect,

4. There must be three days between being part of the grapefruit arm and control arm of this study

5. On day of collect, consume supplied 500 ml apple juice 20 min before first collect

6. Swallow, not chew, peppermint oil capsule with a small amount of water

7. Breath samples will be taken at multiple points across the next 3-4 hrs

For the inhibitor arm, the person was asked to:

1. Day 1, 3 days before sampling, consume half a carton (500 ml) of supplied grapefruit juice

2. Day 2, 2 days before sampling, consume half a carton (500 ml) of supplied grapefruit juice

3. Day 3, 1 day before sampling, consume full carton (1 L) of supplied grapefruit juice.

4. Ahead of each breath collection

a) Fast at least 8 hrs prior to giving first sample. Water can be consumed

b) Not brush teeth within 2 hrs of giving first sample

5. Day 4, Sampling day 1, half carton (500 ml) of supplied grapefruit juice 1 hour before sampling (finish carton later in the day, if performing another grapefruit arm collect the following day)

6. On day of breath collection

a) Swallow, not chew, peppermint oil capsule with a small amount of water

b) Breath samples will be taken at multiple points across the next 3-4 hrs

Test consisted of
taking a 200 mg peppermint capsule.
Collecting breath samples at a series of time points

TABLE 2

| Minutes from start | Action |
|---|---|
| −10 | Control collection |
| 0 | Ingest substrate |
| 10 | Breath collection |
| 20 | Breath collection |
| 30 | Breath collection |
| 40 | Breath collection |
| 50 | Breath collection |
| 60 | Breath collection |
| 75 | Breath collection |
| 90 | Breath collection |
| 115 | Breath collection |
| 140 | Breath Collection |
| 180 | Breath Collection |

### REFERENCES

Chang, M., Tybring, G., Dahl, M. L., Gotharson, E., Sagar, M., Seensalu, R., and Bertilsson, L. (1995). Interphenotype differences in disposition and effect on gastrin levels of omeprazole--suitability of omeprazole as a probe for CYP2C19. Br. J. Clin. Pharmacol. 39, 511-518.

De Kesel, P. M. M., Lambert, W. E., and Stove, C. P. (2016). Alternative Sampling Strategies for Cytochrome CYP450 Phenotyping. Clin. Pharmacokinet. 55, 169-184.

Lin, Y. S., Lockwood, G. F., Graham, M. A., Brian, W. R., Loi, C. M., Dobrinska, M. R., Shen, D. D., Watkins, P. B., Wilkinson, G. R., Kharasch, E. D., et al. (2001). In-vivo phenotyping for CYP3A by a single-point determination of midazolam plasma concentration. Pharmacogenetics 11, 781-791.

Ned Mmsc Phd, R. M. (2010). Genetic testing for CYCYP450 polymorphisms to predict response to clopidogrel: current evidence and test availability. Application: pharmacogenomics. PLoS Curr. 2.

Ou-Yang, D. S., Huang, S. L., Wang, W., Xie, H. G., Xu, Z. H., Shu, Y., and Zhou, H. H. (2000). Phenotypic polymorphism and gender-related differences of CYP1A2 activity in a Chinese population. Br. J. Clin. Pharmacol. 49, 145-151.

Pijls, K. E., de Vries, H., Nikkessen, S., Bast, A., Wodzig, W. K. W. H., and Koek, G. H. (2014). Critical appraisal of 13 C breath tests for microsomal liver function: aminopyrine revisited. Liver Int. 34, 487-494.

Sachse, C., Brockmoller, J., Bauer, S., and Roots, I. (1997). Cytochrome CYP450 2D6 variants in a Caucasian population: allele frequencies and phenotypic consequences. Am. J. Hum. Genet. 60, 284-295.

Samer, C. F., Lorenzini, K. I., Rollason, V., Daali, Y., and Desmeules, J. A. (2013). Applications of CYCYP450 testing in the clinical setting. Mol. Diagn. Ther. 17, 165-184.

Tanaka, E., Kurata, N., and Yasuhara, H. (2003). How useful is the &quot;cocktail approach&quot; for evaluating human hepatic drug metabolizing capacity using cytochrome CYP450 phenotyping probes in vivo? J. Clin. Pharm. Ther. 28, 157-165.

Thacker, D. L., Modak, A., Flockhart, D. A., and Desta, Z. (2012). Is (+)-[13 C]-pantoprazole better than (±)-[13 C]-pantoprazole for the breath test to evaluate CYP2C19 enzyme activity? J. Breath Res. 7, 16001.

Thakur, M., Grossman, I., McCrory, D. C., Orlando, L. A., Steffens, D. C., Cline, K. E., Gray, R. N., Farmer, J., DeJesus, G., O'Brien, C., et al. (2007). Review of evidence for genetic testing for CYCYP450 polymorphisms in management of patients with nonpsychotic depression with selective serotonin reuptake inhibitors. Genet. Med. 9, 826-835.

Zanger, U. M., and Schwab, M. (2013). Cytochrome CYP450 enzymes in drug metabolism: Regulation of gene expression, enzyme activities, and impact of genetic variation. Pharmacol. Ther. 138, 103-141.

1. A method for determining the metabolic activity of a liver enzyme comprising at least one of measuring the concentration of a substrate for a liver enzyme and measuring the concentration of a metabolite of said substrate in exhaled breath of a subject wherein at least one of said substrate and its metabolite is a VOC and wherein the substrate is a non-isotope labelled GRAS compound.

2. The method of claim 1 wherein the metabolic activity is determined from the measured concentration by comparison with one or more reference value.

3. The method of claim 1 wherein the method includes measuring the concentration of a second compound that has substantially the same absorption characteristics as the substrate.

4. The method of claim 1 wherein said liver enzyme is a CYP450 enzyme.

5. The method of claim 1 wherein the GRAS compound is a food or food additive.

6. The method of claim 5 wherein the GRAS compound is a vitamin, phenolic flavoring agent, natural oil, alcohol, amino acid or antioxidant.

7. The method of claim 1 wherein the GRAS compound is an aliphatic or aromatic terpene hydrocarbon or a terpenoid, preferably wherein the GRAS compound is limonene or eucalyptol.

8. (canceled)

9. The method of claim 3 wherein the CYP450 enzyme is selected from at least one of a CYP1 family enzyme, a CYP2 family enzyme, and a CYP3 family enzyme, preferably wherein the CYP450 enzyme is selected from CYP1A1, CYP1A2, CYP1B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7, CYP3A43.

10. (canceled)

11. The method of claim 1 wherein the concentration of two or more metabolites of a single substrate for a liver enzyme is measured, or
wherein the concentration of two or more metabolites of two or more substrates for a liver enzyme is measured, or
wherein the concentration of two or more substrates for a liver enzyme is measured.

12.-13. (canceled)

14. The method of claim 1 wherein the method further includes the step of measuring the concentration of the substrate and/or the metabolite of the substrate in a blood sample of said subject.

15. The method of claim 4 wherein the method further includes the step of determining the CYP450 genotype of said subject.

16. The method of claim 1 wherein the method further includes the step of measuring the concentration of the substrate and/or metabolite in response to a modulator of a liver enzyme, optionally wherein the substrate is formulated as a liquid or tablet or capsule for fast release.

17. (canceled)

18. A method for determining the therapeutically effective dosage of a drug or determining the toxicity of a xenobiotic comprising a method according to claim 1.

19. A method for determining whether a subject suffers from a disorder associated with a liver disease comprising a method according to claim 1.

20. A method for selecting a class of therapeutic compounds or a therapeutic compound comprising a method according to claim 1.

21. A system for determining metabolic activity of a liver enzyme comprising at least one of measuring the concentration of a substrate for a liver enzyme and measuring the concentration of a metabolite of said substrate in exhaled breath of a subject wherein at least one of said substrate and its metabolite is a VOC wherein said system comprises a device for capturing a breath sample from a patient and wherein the substrate is a non-isotope labelled GRAS compound.

22. A system according to claim 21 wherein the liver enzyme is a CYP450 enzyme.

23. A kit comprising a system according to claim 22 and a composition comprising a substrate for a liver enzyme wherein said substrate or its metabolite is a VOC.

24. A kit according to claim 23 wherein said composition is for oral or parenteral administration, optionally wherein the composition is formulated as a liquid or tablet or capsule for fast release.

25. (canceled)

26. A kit according to claim 23 wherein the kit or composition further comprises a second exogenous compound that has substantially the same absorption characteristics as the substrate, optionally wherein the liver enzyme is a CYP450 enzyme.

27. (canceled)
